(19) United States
(12) Patent Application Publication
(10) Pub. No.: US 2024/0212825 A1
(43) Pub. Date: Jun. 27, 2024

(54) USER ABILITY-BASED PERSONALIZED COGNITIVE TRAINING TASK RECOMMENDATION METHOD AND SYSTEM

(71) Applicant: BEIJING WISPIRIT TECHNOLOGY CO., LTD, Beijing (CN)

(72) Inventors: Xiaoyi WANG, Beijing (CN); Zhiming BIAN, Beijing (CN); Zheng CHU, Beijing (CN)

(73) Assignee: BEIJING WISPIRIT TECHNOLOGY CO., LTD, Beijing, BJ (CN)

(21) Appl. No.: 18/420,648

(22) Filed: Jan. 23, 2024

Related U.S. Application Data
(63) Continuation of application No. PCT/CN2022/107482, filed on Jul. 22, 2022.

(30) Foreign Application Priority Data
Jul. 23, 2021 (CN) 202110833758.3

(51) Int. Cl. G16H 20/70; G16H 50/50

(52) U.S. Cl. CPC G16H 20/70; G16H 50/50

(57) ABSTRACT

Disclosed in the present invention are a user ability-based personalized cognitive training task recommendation method and system. The method comprises the following steps: establishing a machine initial test-based recommended training task list; establishing a manual evaluation-based recommended training task list; and merging and sorting to establish an optimal recommended training task list. According to the present invention, personalized cognitive training task recommendation can be performed on the basis of the user's ability, and mutual complementation of treatment schemes is achieved by combining a machine algorithm and manual evaluation, thereby balancing machine and human problems, and reducing decision-making errors.

### BACKGROUND

### Technical Field

The present disclosure relates to a user ability-based personalized cognitive training task recommendation method, also relates to a corresponding personalized cognitive training task recommendation system, and belongs to the technical field of personalized recommendation.

### Related Art

As an important information filtering technology and means, personalized recommendation technology has attracted a large number of scholars for research. At present, this recommendation technology has been widely used in various large-scale multimedia and e-commerce websites, such as Amazon, JD.com, Google News and Taobao. Personalized recommendation technology is mainly divided into three categories: content-based recommendation, collaborative filtering-based recommendation, and hybrid-based recommendation. The collaborative filtering-based recommendation is the most widely used method.

In order to promptly and accurately recommend cognitive training that meets personalized needs to users, researchers have tried to introduce personalized recommendation technology into the medical rehabilitation industry. For example, some researchers have proposed a disease-based personalized recommendation technology. This technology may manually determine the disease status of the users and then recommend training tasks for them. However, existing cognitive training recommendation methods do not take into account the specific impairment of cognitive functions of the users, so there is still a lot of room for improvement in accuracy.

### SUMMARY

A primary technical problem to be solved by the present disclosure is to provide a user ability-based personalized cognitive training task recommendation method, which is appropriate in design, comprehensively considers multiple factors and is high in recommendation result accuracy.

Another technical problem to be solved by the present disclosure is to provide a user ability-based personalized cognitive training task recommendation system.

In order to achieve the above objectives, the present disclosure adopts the following technical solutions:

In a first aspect, an embodiment of the present disclosure provides a user ability-based personalized cognitive training task recommendation method, which includes the following steps:
establishing a recommended task training list of machine preliminary test;
establishing a recommended task training list of manual evaluation; and
performing combination sorting to establish an optimal recommended task training list.

The establishing a recommended task training list of machine preliminary test includes the following steps:
S1-1: establishing a general cognitive capability model, establishing a capability weight matrix (L×K) and a training task capability weight matrix (T×K) according to the general cognitive capability model, and then performing standardization processing on weights in the capability weight matrix and the training task capability weight matrix, where
a formula of establishing a general cognitive capability structural equation model is as follows:

$$x = Λ X η LK + ε LK \\ y = Λ y η TK + ε TK$$

where x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; εLK represents an error on scale measurement; y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; εTK represents an error on training task calculation,
for the capability weight matrix (L×K), L represents a scale set, K represents a capability set; wmn represents the weight of the scale lm∈L to the capability kn∈K, and wmn∈L×K, and
for the training task capability weight matrix (T×K), T represents a training task set, K represents a capability set, rfj represents the weight of the training task tf∈T to the capability kj∈K, and rfj∈T×K;
S1-2: performing test through a preset scale in a system, collecting data according to the answer condition of the scale, and establishing an association score wuk of a user u and a capability k,
S1-3: calculating a matching degree Pui of the user u to a training task i according to the following formula:

$$Pui = \sum Wukrfj \\ k ∈ G ( u , i )$$

where G(u,i) represents the common capability of the user u and the training task i, Wuk represents the association score of the user u and the capability k, and rfj represents the weight of the training task tf∈T to the capability kj∈K; and
S1-4: establishing the recommended task training list of machine preliminary test: sorting all training tasks in a set I(u) according to the user matching degree, where
the set I(u) represents a set of all task training for the user in a current system; and the set will be continuously increased along with system upgrade, for example, 77 task training is provided for a 2.0 system.

The establishing a recommended task training list of manual evaluation includes the following steps:
S2-1: establishing a disease training model: determining an association degree wid between the training task i and a disease d, establishing a disease training task weight matrix
(I×D) and carrying out standardization processing, where
I represents a training task set, D represents a disease set, wid represents the weight of the training task i∈I to the disease d∈D, and wid∈I×D;
S2-2: determining an association score Qui of the user u and the disease d based on a medical detection result held by the user, the score range being 1-100, and du∈D;
S2-3: calculating a matching degree P′ui of the user u to the training task i according to the following formula:

$${P}^{′} ui = \sum QuiWid \\ d ∈ H ( u )$$

where H(u) represents a set of diagnosed diseases of the user, Wid represents the association score of the training task i and the disease d, and Qui represents the association score of the user u and the disease d, and
S2-4: establishing the recommended task training list of manual evaluation: carrying out weighting, de-weighting and sorting on all training tasks in a set I′(u) according to the user matching degree, where
the set I′(u) refers to a set of general task training corresponding to diseases based on literatures or experience, and may be a union set of a plurality of diseases; the set will be continuously changed along with deepening of cognition to disease, for example, the trainable tasks for hemineglect include High Altitude Bird Catching, One-Decision, and Branch Searching and Fruit Picking.

The establishing an optimal recommended task training list includes the steps of: performing dual evaluation recommendation on the training task; performing weighting and summing on the matching degrees Pui and P′ui of the user u to the training task i respectively; and calculating a recommendation score S of each training task,

$$S = aPui + {bP}^{′} ui$$

where a represents the weight corresponding to machine preliminary test; and b represents the weight corresponding to manual evaluation, the training tasks are arranged according to the scores, and the score arrangement sequence is a sequence of the recommended training tasks.

Preferably, the association score wuk of the user u and the capability k is established through the following steps:
sequentially implementing n preset comprehensive evaluation subunits of the machine preliminary test by the user and respectively generating a raw score Xn;
extracting the raw score of a subject in the n comprehensive evaluation subunits, and carrying out standardized conversion on the raw scores in the comprehensive evaluation subunits according to comprehensive norm parameters of a healthy user, so as to generate the association score wuk of the user u and the capability K; the formula is as follows:

$$Wuk = wmn * \{ 100 - 10 * ( Xi - \overline{X} i ) / σ i \}$$

where i ranges from 1−n; Wuk represents scores of the comprehensive evaluation subunits after the standardized conversion, namely the association scores of the user u and the capability K; if the measured capability of the user is relatively poor, the more the deviation from the norm is, the larger the value of Wuk is; Xi represents the raw scores in the comprehensive evaluation subunits; X̅i represents an average value of the raw scores of the comprehensive evaluation subunits of healthy people matched with the subject by age, gender, profession and education degree; σi represents a standard deviation of the raw scores of the comprehensive evaluation subunits of the healthy people matched with the subject by age, gender, profession and education degree; X̅i and σi are also referred to as the comprehensive norm parameters of the healthy user; and wmn represents the weight of the scale lm∈L to the capability kn∈K.

In a second aspect, an embodiment of the present disclosure provides a user ability-based personalized cognitive training task recommendation system, which includes a machine preliminary test module, a manual evaluation module and a comprehensive recommendation module.

The machine preliminary test module is configured to establish a recommended task training list of machine preliminary test, and the recommended task training list of machine preliminary test is established through the following steps:
S1-1: establishing a general cognitive capability model, establishing a capability weight matrix (L×K) and a training task capability weight matrix (T×K) according to the general cognitive capability model, and then performing standardization processing on weights in the capability weight matrix and the training task capability weight matrix, where
a formula of establishing a general cognitive capability structural equation model is as follows:

$$x = Λ X η LK + ε LK \\ y = Λ y η TK + ε TK$$

where x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; εLK represents an error on scale measurement; y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; εTK represents an error on training task calculation,
for the capability weight matrix (L×K), L represents a scale set, K represents a capability set; wmn represents the weight of the scale lm∈L to the capability kn∈K, and wmn∈L×K, and
for the training task capability weight matrix (T×K), T represents a training task set, K represents a capability set, rfj represents the weight of the training task tf∈T to the capability kj∈K, and rfj∈T×K;
S1-2: performing test through a preset scale, collecting data according to the answer condition of the scale, and establishing an association score wuk of a user u and a capability k;
S1-3: calculating a matching degree Pui of the user u to a training task i according to the following formula:

$$Pue = \sum Wukrfj$$

$$k ∈ G ( u , i )$$

where G(u,i) represents the common capability of the user u and the training task i, Wuk represents the association score of the user u and the capability k, and rfj represents the weight of the training task tf∈T to the capability kj∈K; and
S1-4: establishing the recommended task training list of machine preliminary test: sorting all training tasks in a set I(u) according to the user matching degree, the set I(u) representing a set of all task training for the user in a current system.

The manual evaluation module is configured to establish a recommended task training list of manual evaluation, and the recommended task training list of manual evaluation is established through the following steps:
S2-1: establishing a disease training model, determining an association degree wid between the training task i and a disease d, establishing a disease training task weight matrix (I×D) and carrying out standardization processing, where
I represents a training task set, D represents a disease set, wid represents the weight of the training task i∈I to the disease d∈D, and wid∈I×D;
S2-2: determining an association score Qui of the user u and the disease d based on a medical detection result held by the user, the score range being 1-100, and du∈D;
S2-3: calculating a matching degree P′ui of the user u to the training task i according to the following formula:

$${P}^{′} u i = \sum Qui Wid$$

$$d ∈ H ( u )$$

where H(u) represents a set of diagnosed diseases of the user, Wid represents the association score of the training task i and the disease d, and Qui represents the association score of the user u and the disease d, and
S2-4: establishing the recommended task training list of manual evaluation, and carrying out weighting, de-weighting and sorting on all training tasks in a set I′(u) according to the user matching degree.

The comprehensive recommendation module is configured to establish an optimal recommended task training list, and the optimal recommended task training list is established through the following steps:
performing dual evaluation recommendation on the training task; performing weighting and summing on the matching degrees Pui and P′ui of the user u to the training task i respectively; and calculating a recommendation score S of each training task,

$$S = a P u i + b {P}^{′} u i$$

where a represents the weight corresponding to machine preliminary test, and b represents the weight corresponding to manual evaluation, the training tasks are arranged according to scores, and the score arrangement sequence is a sequence of the recommended training tasks.

According to the present disclosure, the accuracy of the cognitive training recommendation method is improved through algorithm optimization; cognitive training tasks can be recommended in a personalizing way based on the user capabilities; and treatment solutions are complementary to each other in a mode of combining the machine algorithm with manual evaluation, so that the machine and manual problems are balanced and decision errors are reduced. The present disclosure has relatively high compatibility and expansibility based on continuous enrichment of cognitive task training.

### BRIEF DESCRIPTION OF THE DRAWINGS

FIG. 1 is a flowchart of a user ability-based personalized cognitive training task recommendation method according to an embodiment of the present disclosure;

FIG. 2 is an instance diagram of constructing cognitive capability according to an embodiment of the present disclosure;

FIG. 3 is another instance diagram of constructing cognitive capability according to an embodiment of the present disclosure; and

FIG. 4 is a schematic structural diagram of a user ability-based personalized cognitive training task recommendation system according to an embodiment of the present disclosure.

### DETAILED DESCRIPTION

The technical content of the present disclosure will be described in detail below with reference to the accompanying drawings and specific embodiments.

In order to make the objective, technical solutions and advantages of the present disclosure more clear, the technical solutions of the present disclosure will be clearly and completely described in detail below in combination with the specific embodiments and corresponding accompanying drawings of the present disclosure. Obviously, the described embodiments are only part of the embodiments of the present disclosure, not the whole embodiment. All other embodiments obtained by a person of ordinary skill in the art based on the disclosed embodiments without creative efforts shall fall within the protection scope of the present disclosure.

A user ability-based personalized cognitive training task recommendation method provided by an embodiment of the present disclosure includes at least the following steps:
establish a recommended task training list of machine preliminary test;
establish a recommended task training list of manual evaluation; and
perform combination sorting to establish an optimal recommended task training list.

The establishing the recommended task training list of machine preliminary test includes the following steps:
S1-1: a general cognitive capability model is established, a capability weight matrix (L×K) and a training task capability weight matrix (T×K) are established according to the general cognitive capability model, and then standardization processing is performed on weights in the capability weight matrix and the training task capability weight matrix, where
a formula of establishing a general cognitive capability structural equation model is as follows:

$$x = Λ X η L K + ε LK$$

$$y = Λ y η TK + ε T K$$

where x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; εLK represents an error on scale measurement; y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; εTK represents an error on training task calculation,
for the capability weight matrix (L×K), L represents a scale set, K represents a capability set; wmn represents the weight of the scale lm∈L to the capability kn∈K, and wmn∈L×K, and
for the training task capability weight matrix (T×K), T represents a training task set, K represents a capability set, rfj represents the weight of the training task tf∈T to the capability kj∈K, and rfj∈T×K;
S1-2: a test is performed through a preset scale in a system, data is collected according to the answer condition of the scale, and an association score wuk of a user u and a capability k is established,
S1-3: a matching degree Pui of the user u to a training task i is calculated according to the following formula:

$$Pui = \sum Wukrfj$$

$$k ∈ G ( u , i )$$

where G(u,i) represents the common capability of the user u and the training task i, Wuk represents the association score of the user u and the capability k, and rfj represents the weight of the training task tf∈T to the capability kj∈K; and
S1-4: the recommended task training list of machine preliminary test is established: all training tasks in a set I(u) are sorted according to the user matching degree;
the set I(u) represents a set of all task training for the user in a current system; and the set will be continuously increased along with system upgrade, for example, 77 task training is provided for a 2.0 system.

The establishing the recommended task training list of manual evaluation includes the following steps:
S2-1: a disease training model is established: an association degree wid between the training task i and a disease d is determined, a disease training task weight matrix (I×D) is established and standardization processing is carried out, where
I represents a training task set, D represents a disease set,
wid represents the weight of the training task i∈I to the disease d∈D, and wid∈I×D;
S2-2: an association score Qui of the user u and the disease d is determined based on a medical detection result held by the user, the score range is 1-100, and du∈D;
S2-3: a matching degree P′ui of the user u to the training task i is calculated according to

$${P}^{′} u i = \sum QuiWid$$

$$d ∈ H ( u )$$

where H(u) represents a set of diagnosed diseases of the user, Wid represents the association score of the training task i and the disease d, and Qui represents the association score of the user u and the disease d.
S2-4: the recommended task training list of manual evaluation is established: weighting, de-weighting and sorting are carried out on all training tasks in a set I′(u) according to the user matching degree;
the set I′(u) refers to a set of general task training corresponding to diseases based on literatures or experience, and may be a union set of a plurality of diseases; the set will be continuously changed along with deepening of cognition to disease, for example, the trainable tasks for hemineglect include High Altitude Bird Catching, One-Decision, and Branch Searching and Fruit Picking.

The establishing the optimal recommended task training list includes the steps of: dual evaluation recommendation is performed on the training task; the matching degrees Pui and P′ui of the user u to the training task i are weighted and then summed; and a recommendation score S of each training task is calculated,

$$S = a P u i + b {P}^{′} u i$$

where a represents the weight corresponding to machine preliminary test; and b represents the weight corresponding to manual evaluation, the training tasks are arranged according to scores, and the sequence rule is the optimal recommendation.

The system defaults of weights “a” and “b” are 0.5 and 0.5, which indicates that task training recommendation respects double opinions of a machine algorithm and manual evaluation; if a=1 and b=0, it completely refers to the recommendation of the machine algorithm; and if a=0 and b=1, it completely refers to the recommendation of the manual evaluation. In practical application, it can be set by an experienced therapist according to the understanding on a patient, and the machine algorithm and manual evaluation are complementary to each other, so that the decision error of a single machine algorithm or manual evaluation is reduced.

According to the user ability-based personalized cognitive training task recommendation method provided by this embodiment of the present disclosure,
the association score wuk of the user u and the capability k is established through the following steps:
n preset comprehensive evaluation subunits of the machine preliminary test are sequentially implemented by the user and a raw score Xn is respectively generated;
the raw score of a subject in the n comprehensive evaluation subunits is extracted, and standardized conversion is carried out on the raw scores in the comprehensive evaluation subunits according to comprehensive norm parameters of a healthy user, so as to generate the association score wuk of the user u and the capability K; the formula is as follows:

$$Wuk = wmn * \{ 100 - 10 * ( X i - \overline{X} i ) σ i \}$$

where i ranges from 1−n; Wuk represents scores of the comprehensive evaluation subunits after the standardized conversion, namely the association scores of the user u and the capability K; if the measured capability of the user is relatively poor, the more the deviation from the norm is, the larger the value of Wuk is; Xi represents the raw scores in the comprehensive evaluation subunits; X̅i represents an average value of the raw scores of the comprehensive evaluation subunits of healthy people matched with the subject by age, gender, profession and education degree; σi represents a standard deviation of the raw scores of the comprehensive evaluation subunits of the healthy people matched with the subject by age, gender, profession and education degree; X̅i and σi are also referred to as the comprehensive norm parameters of the healthy user; and wmn represents the weight of the scale lm∈L to the capability kn∈K.

The technical solutions provided by various embodiments of the present disclosure will be described in detail below with reference to the accompanying drawings.

As shown in FIG. 1, a user ability-based personalized cognitive training task recommendation method includes at least the following steps:
S1: establish a recommended task training list of machine preliminary test;
S2: establish a recommended task training list of manual evaluation; and
S3: perform combination sorting to establish an optimal recommended task training list.

Step S1 of establishing the recommended task training list of machine preliminary test includes the following steps:
S1-1: a general cognitive capability model is established, a capability weight matrix and a training task capability weight matrix are established according to research achievements of intelligence theory and brain functional networks, and then standardization processing is performed on weights in the capability weight matrix and the training task capability weight matrix.

A formula of establishing a general cognitive capability structural equation model is as follows:

$$x = Λ X η L K + ε LK$$

$$y = Λ y η TK + ε TK$$

where x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; and εLK represents an error on scale measurement. y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; and εTK represents an error on training task calculation.

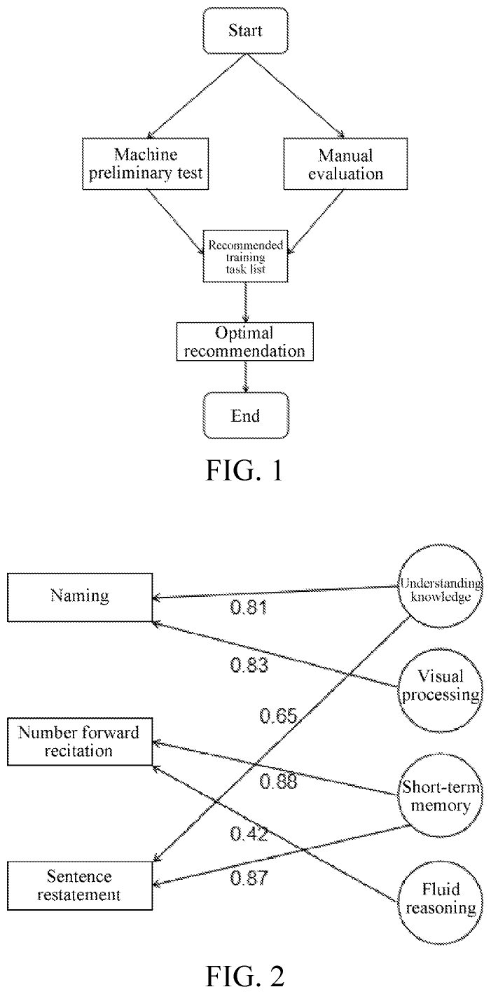

For example, as shown in FIG. 2, in one embodiment of the present disclosure, x BNT is x, and BNT is an abbreviation of a naming sub-test in a MoCA test; ∧X=0.81; ηBNT-Gc is ηLK, BNT is an abbreviation of the naming sub-test in the MoCA test, and Gc is an abbreviation of capability understanding knowledge; εBNT-Gc is εLK, and the value is 0; an expression of the naming sub-test in the MoCA test and the capability understanding knowledge is shown as below:

$$xBNT = 0.81 * ηBNT - G c + ε B N T - G c$$

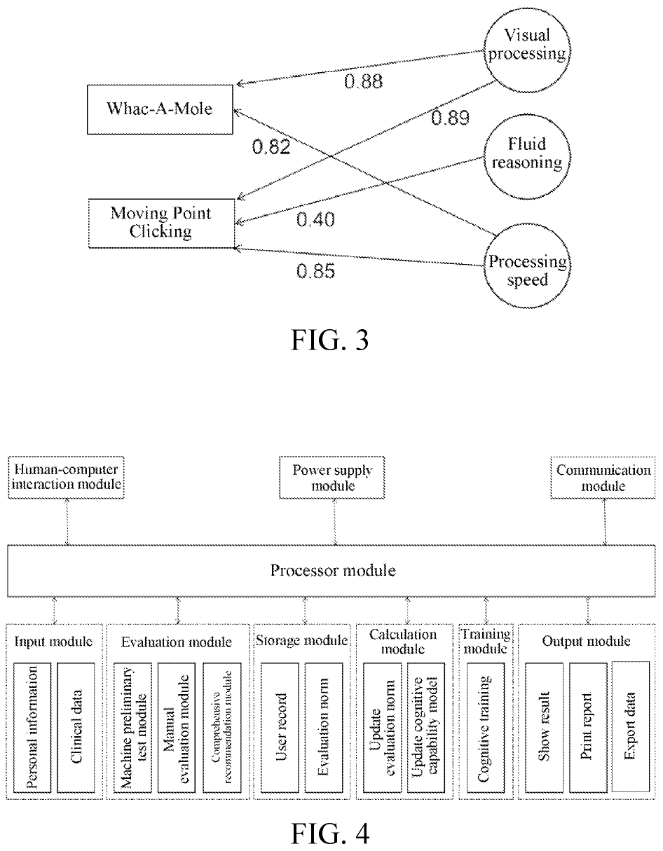

As shown in FIG. 3, in one embodiment of the present disclosure, y 30001 is y, and 30001 is an ID of Whac-A-Mole; ∧X=0.88; η30001−Gv is ηTK, 30001 is the ID of Whac-A-Mole, and Gv is an abbreviation of capability visual processing; and ε30001-Gv is εTK, and the value is 0; and an expression of the Whac-A-Mole and the capability visual processing is shown as below:

$$y 30001 = 0.8 * η30001 - Gv + ε 30 \; 001 - Gv$$

The capability weight matrix (L×K) is established according to the general cognitive capability structural equation model; L represents the scale set; K represents the capability set; and wmn represents the weight of the scale lm∈L to the capability kn∈K, and wmn∈L×K.

TABLE 1
Capability weight matrix

| | Under- | | | Short- | |
|---|---|---|---|---|---|
| | standing | Visual | Fluid | term | Processing |
| | knowledge | processing | reasoning | memory | speed |
| Naming | 0.81 | 0.83 | | | |
| Number | | | 0.42 | 0.88 | |
| forward | | | | | |
| recitation | | | | | |
| Sentence | 0.65 | | | 0.87 | |
| restatement | | | | | |

Note:
The scale set L involves 3 elements of the naming sub-test, the number forward recitation sub-test and the sentence restatement sub-test in the MoCA test. The capability set K involves 5 elements of understanding knowledge, visual processing, fluid reasoning, short-term memory and processing speed.

The training task capability weight matrix (T×K) is established according to the general cognitive capability structural equation model, where
T represents the training task set; K represents the capability set; and rfj represents the weight of the training task tf∈T to the capability kj∈K, and rfj∈T×K.

TABLE 2
Training task capability weight matrix

| | | | | |
|---|---|---|---|---|
| Under- | | | Short- | |
| standing | Visual | Fluid | term | Processing |
| knowledge | processing | reasoning | memory | speed |

| | | | |
|---|---|---|---|
| Whac-A-Mole | 0.88 | | 0.82 |
| Moving Point | 0.89 | 0.4 | 0.85 |
| Clicking | | | |

Note:
The scale set T involves 2 elements of Whac-A-Mole, and Moving Point Clicking. The capability set K involves 5 elements of understanding knowledge, visual processing, fluid reasoning, short-term memory and processing speed.

S1-2: a capability state of the user is calculated through machine preliminary test; the machine preliminary test refers to that a test is performed through a preset scale in the system, data is collected according to the answer condition of the scale, a measurement result can be intelligently analyzed and compared with norm to determine the capability state of the user, and the association score wuk of the user u and the capability k is established. The test performed through the scale preset in the system includes the scales and task type evaluation tools with relatively wide clinical applications such as MoCA, MMSE, ADL, PHQ-9, GAD-7, and life satisfaction indexes.

The association score wuk of the user u and the capability k is established through the following steps:
1, 2, 3 . . . , n evaluations of the comprehensive evaluation subunits preset in the machine preliminary test are sequentially implemented by the user, and raw scores X01, X02, X03 . . . , Xn are generated.

For example, the user Zhang performs MoCA test, a MoCA evaluation score table is generated, and the raw scores are displayed in the last column, including a total score and a sub-item score, as shown in Table 3 below.

TABLE 3
MoCA evaluation score table

| | | Total | Raw |
|---|---|---|---|
| Item | Sub-item | score | score |
| Total score | | 30 | 25 |
| Visual space and | | 5 | 5 |
| executive capability | | | |
| Naming | | 3 | 3 |
| Attention | Number forward recitation | 1 | 0 |
| | Number backward recitation | 1 | 0 |
| | Alertness | 1 | 1 |
| | Continuously reduce by 7 | 3 | 3 |
| Language | | 3 | 3 |
| Abstract capability | | 2 | 2 |
| Delayed memory | | 5 | 2 |
| Directive force | | 6 | 6 |

According to the present disclosure, the recommendation system is capable of extracting the raw scores of the subject in the n comprehensive evaluation subunits, and carrying out standardized conversion on the raw scores in the comprehensive evaluation subunits according to the comprehensive norm parameters of the healthy user, so as to generate the association score wuk of the user u and the capability k; the formula is as follows:

$$Wuk = wmn * \{ 100 - 10 * ( X i - \overline{X} i ) σi \}$$

where i ranges from 1−n;
Wuk represents scores of the comprehensive evaluation subunits after the standardized conversion, namely the association scores of the user u and the capability K; if the measured capability of the user is relatively poor, the more the deviation from the norm is, the larger the value of Wuk is;
Xi represents the raw scores in the comprehensive evaluation subunits;
X̅i represents the average value of the raw scores of the comprehensive evaluation subunits of the healthy people matched with the subject by age, gender, profession and education degree;
σi represents the standard deviation of the raw scores of the comprehensive evaluation subunits of the healthy people matched with the subject by age, gender, profession and education degree;
X̅i and σi are also referred to as the comprehensive norm parameters of the healthy user; and
wmn represents the weight of the scale lm∈L to the capability kn∈K.

In one embodiment of the present disclosure, according to the norm parameters and the calculation formula, the association score of the user Zhang in the number forward recitation sub-test to the fluid reasoning capability in MoCA test is obtained as Wuk=46.2, and the association score to the short-term memory capability is obtained as Wuk=96.8.

S1-3: a matching degree Pui of the user u to the training task i is calculated according to the following formula:

$$Pui = \sum Wukrfj$$

$$k ∈ G ( u , i )$$

where G(u,i) represents the common capability of the user u and the training task i;
Wuk represents the association score of the user u and the capability k; and
rfj represents the weight of the training task tf∈T to the capability kj∈K.

In one embodiment of the present disclosure, the matching degree of the user Zhang to the training task of Whac-A-Mole is obtained as Pui=86.4, and the matching degree to the training task of Moving Point Clicking is obtained as Pui=94.6.

S1-4: the recommended task training list of machine preliminary test is established: all training tasks in the set I(u) are sorted according to the user matching degree. Top-N articles are taken to provide an explanation for each training task. The Top-N training tasks are saved in the recommended task training list of machine preliminary test, for example, the ranking of Moving Point Clicking is higher than that of Whac-A-Mole.

The establishing the recommended task training list of manual evaluation includes the following steps.

S2-1: a disease training model is established, an association degree wid between the training task i and a disease d is determined based on disease pathological characteristics and rehabilitation training literature, a disease training task weight matrix (I×D) is established and standardization processing is carried out. Where
I represents a training task set;
D represents a disease set; and
wid represents the weight of the training task i∈I to the disease d∈D, and wid∈I×D.

TABLE 4
Recommended task training list

| | | Mild | Vascular | |
|---|---|---|---|---|
| | Alzheimer's | cognitive | cognitive | |
| | disease | impairment | impairment | Hemineglect |
| Whac-A-Mole | 0.65 | 0.74 | 0.76 | 0.86 |
| Moving Point | 0.87 | 0.92 | 0.79 | 0.82 |
| Clicking | | | | |

S2-2: the condition of the user (disease) is comprehensively determined according to a doctor diagnosis result (manual evaluation), including the evaluation results based on the subject statement, the medical history, the physical examination condition, the imagological examination result, the daily life capability and other scale evaluation results, and represented with a Qui score from 1 to 100, the higher the score is, the higher the risk of suffering from the disease d is, and du∈D; and in one embodiment of the present disclosure, the diagnosis result shows that the user Zhang has a high risk of suffering from mild cognitive impairment, and Qui=98.

S2-3: a matching degree P′ui of the user u to the training task i is calculated according to the following formula:

$${P}^{′} u i = \sum QuiWid$$

$$d ∈ H ( u )$$

where H(u) represents a set of diagnosed diseases of the user;
Wid represents the association score of the training task i and the disease d; and
Qui represents the association score of the user u and the disease d.

In one embodiment of the present disclosure, the matching degree of the user Zhang to the training task of Whac-A-Mole is obtained as P′ui=72.52, and the matching degree to the training task of Moving Point Clicking is obtained as P′ui=90.16.

S2-4: the recommended task training list of manual evaluation is established: weighting, de-weighting and sorting are carried out on all training tasks in the set I′(u) according to the user matching degree. Top-N articles are taken to provide an explanation for each training task. The Top-N training tasks are saved in the recommended task training list of manual evaluation, for example, the ranking of Moving Point Clicking is higher than that of Whac-A-Mole.

In the steps of “perform combination sorting to establish an optimal recommended task training list”, the training tasks are recommended through dual evaluation, so the user generally accumulates a large number of training tasks. The matching degrees Pui and P′ui of the user u to the training task i are weighted and then summed, and the recommendation score S of each training task is calculated.

$$S = a P u i + bP ' ui$$

where a represents the weight corresponding to machine preliminary test; and b represents the weight corresponding to manual evaluation.

The training tasks are arranged according to scores, and the sequence rule is the optimal recommendation.

In one embodiment of the present disclosure, the recommendation score of the Whac-A-Mole training task for the user Zhang is obtained as S=79.46, and the recommendation score of the Moving Point Clicking training task for the user Zhang is obtained as S=92.38. The ranking of Moving Point Clicking is higher than that of Whac-A-Mole.

As shown in FIG. 4, an embodiment of the present disclosure further provides a user ability-based personalized cognitive training task recommendation system, which includes a processor module, a power supply module, a human-computer interaction module, a communication module, an input module, an evaluation module, a storage module, a calculation module and an output module. The power supply module, the human-computer interaction module, the communication module, the input module, the evaluation module, the storage module, the calculation module and the output module are connected with the processor module, respectively. The evaluation module includes a machine preliminary test module, a manual evaluation module and a comprehensive difficulty recommendation module.

The machine preliminary test module is configured to establish a recommended task training list of machine preliminary test, and the recommended task training list of machine preliminary test is established through the following steps:
S1-1: a general cognitive capability model is established, a capability weight matrix (L×K) and a training task capability weight matrix (T×K) are established according to the general cognitive capability model, and then standardization processing is performed on weights in the capability weight matrix and the training task capability weight matrix, where
a formula of establishing a general cognitive capability structural equation model is as follows:

$$\begin{matrix}x = Λ X η LK + ε L K \\ y = Λ y η TK + ε T K\end{matrix}$$

where x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; εLK represents an error on scale measurement; y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; εTK represents an error on training task calculation,
for the capability weight matrix (L×K), L represents a scale set, K represents a capability set; wmn represents the weight of the scale lm∈L to the capability kn∈K, and wmn∈L×K, and
for the training task capability weight matrix (T×K), T represents a training task set, K represents a capability set, rfj represents the weight of the training task tf∈T to the capability kj∈K, and rfj∈T×K;
S1-2: a test is performed through a preset scale, data is collected according to the answer condition of the scale, and an association score wuk of a user u and a capability k is established;
S1-3: a matching degree Pui of the user u to a training task i is calculated according to the following formula:

$$\begin{matrix}Pui = \sum Wukrfj \\ k ∈ G ( u , i )\end{matrix}$$

where G(u,i) represents the common capability of the user u and the training task i, Wuk represents the association score of the user u and the capability k, and rfj represents the weight of the training task tf∈T to the capability kj∈K; and
S1-4: the recommended task training list of machine preliminary test is established: all training tasks in a set I(u) are sorted according to the user matching degree, and the set I(u) represents a set of all task training for the user in a current system.

The manual evaluation module is configured to establish a recommended task training list of manual evaluation, and the recommended task training list of manual evaluation is established through the following steps:
S2-1: a disease training model is established, an association degree wid between the training task i and a disease d is determined, a disease training task weight matrix (I×D) is established and standardization processing is carried out, where
I represents a training task set, D represents a disease set,
wid represents the weight of the training task i∈I to the disease d∈D, and wid∈I×D;
S2-2: an association score Qui of the user u and the disease d is determined based on a medical detection result held by the user, the score range is 1-100, and du∈D;
S2-3: a matching degree P′ui of the user u to the training task i is calculated according to the following formula:

$$\begin{matrix}P ' ui = \sum QuiWid \\ d ∈ H ( u )\end{matrix}$$

where H(u) represents a set of diagnosed diseases of the user, Wid represents the association score of the training task i and the disease d, and Qui represents the association score of the user u and the disease d, and
S2-4: the recommended task training list of manual evaluation is established, and weighting, de-weighting and sorting are carried out on all training tasks in a set I′(u) according to the user matching degree.

The comprehensive recommendation module is configured to establish an optimal recommended task training list, and the optimal recommended task training list is established through the following steps:
dual evaluation recommendation is performed on the training task; the matching degrees Pui and P′ui of the user u to the training task i are weighted and then summed; and a recommendation score S of each training task is calculated,

$$S = a P u i + bP ' ui$$

where a represents the weight corresponding to machine preliminary test, and b represents the weight corresponding to manual evaluation, the training tasks are arranged according to scores, and the sequence rule is the optimal recommendation.

The foregoing are merely specific embodiments of the present disclosure, and are not intended to limit the present disclosure. For those of skill in the art, the present disclosure can have various changes and variations. Any modification, equivalent replacement, improvement and the like made within the principle of the present disclosure should be included in the protection scope of the claims of the present disclosure.

What is claimed is:

1. A user ability-based personalized cognitive training task recommendation method, comprising the following steps:
establishing a recommended task training list of machine preliminary test;
establishing a recommended task training list of manual evaluation; and
performing combination sorting to establish an optimal recommended task training list.

2. The user ability-based personalized cognitive training task recommendation method according to claim 1, wherein the establishing a recommended task training list of machine preliminary test comprises the following steps:
S1-1: establishing a general cognitive capability model, establishing a capability weight matrix (L×K) and a training task capability weight matrix (T×K) according to the general cognitive capability model, and then performing standardization processing on weights in the capability weight matrix and the training task capability weight matrix;
a formula of establishing a general cognitive capability structural equation model is as follows:
$$\begin{matrix}x = Λ X η LK + ε L K \\ y = Λ y η TK + ε T K\end{matrix}$$
wherein x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; εLK represents an error on scale measurement; y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; εTK represents an error on training task calculation,
for the capability weight matrix (L×K), L represents a scale set, K represents a capability set; wmn represents the weight of the scale lm∈L to the capability kn∈K, and wmn∈L×K, and
for the training task capability weight matrix (T×K), T represents a training task set, K represents a capability set, rfj represents the weight of the training task tf∈T to the capability kj∈K, and rfj∈T×K;
S1-2: performing test through a preset scale, collecting data according to the answer condition of the scale, and establishing an association score wuk of a user u and a capability k;
S1-3: calculating a matching degree Pui of the user u to a training task i according to the following formula:
$$\begin{matrix}Pui = \sum Wukrfj \\ k ∈ G ( u , i )\end{matrix}$$
wherein G(u,i) represents the common capability of the user u and the training task i, Wuk represents the association score of the user u and the capability k, and rfj represents the weight of the training task tf∈T to the capability kj∈K; and
S1-4: establishing the recommended task training list of machine preliminary test: sorting all training tasks in a set I(u) according to the user matching degree, the set I(u) representing a set of all task training for the user in a current system.

3. The user ability-based personalized cognitive training task recommendation method according to claim 1, wherein the establishing a recommended task training list of manual evaluation comprises the following steps:
S2-1: establishing a disease training model, determining an association degree wid between the training task i and a disease d, establishing a disease training task weight matrix (I×D) and carrying out standardization processing, wherein
I represents a training task set, D represents a disease set,
wid represents the weight of the training task i∈I to the disease d∈D, and wid∈I×D;
S2-2: determining an association score Qui of the user u and the disease d based on a medical detection result held by the user, the score range being 1-100, and du∈D;
S2-3: calculating a matching degree P′ui of the user u to the training task i according to the following formula:
$$\begin{matrix}P ' ui = \sum QuiWid \\ d ∈ H ( u )\end{matrix}$$
wherein H(u) represents a set of diagnosed diseases of the user, Wid represents the association score of the training task i and the disease d, and Qui represents the association score of the user u and the disease d, and
S2-4: establishing the recommended task training list of manual evaluation, and carrying out weighting, de-weighting and sorting on all training tasks in a set I′(u) according to the user matching degree.

4. The user ability-based personalized cognitive training task recommendation method according to claim 1, wherein the establishing an optimal recommended task training list comprises the following steps:
performing dual evaluation recommendation on the training task; performing weighting and summing on the matching degrees Pui and P′ui of the user u to the training task i respectively; and calculating a recommendation score S of each training task,
$$S = aP u i + bP ' ui$$
wherein a represents the weight corresponding to machine preliminary test, and b represents the weight corresponding to manual evaluation, the training tasks are arranged according to scores, and the score arrangement sequence is a sequence of the recommended training tasks.

5. The user ability-based personalized cognitive training task recommendation method according to claim 1, wherein the association score wuk of the user u and the capability k is established through the following steps:
sequentially implementing n preset comprehensive evaluation subunits of the machine preliminary test by the user and respectively generating a raw score Xn;
extracting the raw score of a subject in the n comprehensive evaluation subunits, and carrying out standardized conversion on the raw scores in the comprehensive evaluation subunits according to comprehensive norm parameters of a healthy user, so as to generate the association score wuk of the user u and the capability K; the formula is as follows:
$$Wuk = wmn * \{ 100 - 10 * ( Xi - \overline{X} i ) / σ i \}$$
wherein i ranges from 1−n; Wuk represents scores of the comprehensive evaluation subunits after the standardized conversion, namely the association scores of the user u and the capability K; if the measured capability of the user is relatively poor, the more the deviation from the norm is, the larger the value of Wuk is; Xi represents the raw scores in the comprehensive evaluation subunits; X̅i represents an average value of the raw scores of the comprehensive evaluation subunits of healthy people matched with the subject by age, gender, profession and education degree; σi represents a standard deviation of the raw scores of the comprehensive evaluation subunits of the healthy people matched with the subject by age, gender, profession and education degree; X̅i and σi are also referred to as the comprehensive norm parameters of the healthy user; and wmn represents the weight of the scale lm∈L to the capability kn∈K.

6. A user ability-based personalized cognitive training task recommendation system, comprising a machine preliminary test module, a manual evaluation module and a comprehensive recommendation module, wherein
the machine preliminary test module is configured to establish a recommended task training list of machine preliminary test;
the manual evaluation module is configured to establish a recommended task training list of manual evaluation; and
the comprehensive recommendation module is configured to establish an optimal recommended task training list.

7. The user ability-based personalized cognitive training task recommendation system according to claim 6, wherein the machine preliminary test module is configured to establish the recommended task training list of machine preliminary test through the following steps:
S1-1: establishing a general cognitive capability model, establishing a capability weight matrix (L×K) and a training task capability weight matrix (T×K) according to the general cognitive capability model, and then performing standardization processing on weights in the capability weight matrix and the training task capability weight matrix, wherein
a formula of establishing a general cognitive capability structural equation model is as follows:
$$\begin{matrix}x = Λ X η LK + ε L K \\ y = Λ y η TK + ε T K\end{matrix}$$
wherein x represents a vector consisting of sub-items of a single-item scale or a comprehensive scale; ηLK represents a vector consisting of cognitive capabilities; ∧X represents a relationship between the scale and the cognitive capabilities and is a factor load matrix of the scale on the cognitive capabilities; εLK represents an error on scale measurement; y represents a vector consisting of training tasks; ηTK represents a vector consisting of the cognitive capabilities; ∧y represents a relationship between the training tasks and the cognitive capabilities and is a factor load matrix of the training tasks on the cognitive capabilities; εTK represents an error on training task calculation,
for the capability weight matrix (L×K), L represents a scale set, K represents a capability set; wmn represents the weight of the scale lm∈L to the capability kn∈K, and wmn∈L×K, and
for the training task capability weight matrix (T×K), T represents a training task set, K represents a capability set, rfj represents the weight of the training task tf∈T to the capability kj∈K, and rfj∈T×K;
S1-2: performing test through a preset scale, collecting data according to the answer condition of the scale, and establishing an association score wuk of a user u and a capability k;
S1-3: calculating a matching degree Pui of the user u to a training task i according to the following formula:
$$\begin{matrix}Pui = \sum Wukrfj \\ k ∈ G ( u , i )\end{matrix}$$
where G(u,i) represents the common capability of the user u and the training task i, Wuk represents the association score of the user u and the capability k, and rfj represents the weight of the training task tf∈T to the capability kj∈K; and
S1-4: establishing the recommended task training list of machine preliminary test: sorting all training tasks in a set I(u) according to the user matching degree, the set I(u) representing a set of all task training for the user in a current system.

8. The user ability-based personalized cognitive training task recommendation system according to claim 6, wherein the manual evaluation module is configured to establish the recommended task training list of manual evaluation through the following steps:
S2-1: establishing a disease training model, determining an association degree wid between the training task i and a disease d, establishing a disease training task weight matrix (I×D) and carrying out standardization processing, wherein
I represents a training task set, D represents a disease set,
wid represents the weight of the training task i∈I to the disease d∈D, and wid∈I×D;
S2-2: determining an association score Qui of the user u and the disease d based on a medical detection result held by the user, the score range being 1-100, and du∈D;
S2-3: calculating a matching degree P′ui of the user u to the training task i according to the following formula:
$$\begin{matrix}P ' ui = \sum QuiWid \\ d ∈ H ( u )\end{matrix}$$
wherein H(u) represents a set of diagnosed diseases of the user, Wid represents the association score of the training task i and the disease d, and Qui represents the association score of the user u and the disease d, and
S2-4: establishing the recommended task training list of manual evaluation, and carrying out weighting, de-weighting and sorting on all training tasks in a set I′(u) according to the user matching degree.

9. The user ability-based personalized cognitive training task recommendation system according to claim 6, wherein the comprehensive recommendation module is configured to establish the optimal recommended task training list through the following steps:
performing dual evaluation recommendation on the training task; performing weighting and summing on the matching degrees Pui and P′ui of the user u to the training task i respectively; and calculating a recommendation score S of each training task,
$$S = aPu i + bP ' ui$$
wherein a represents the weight corresponding to machine preliminary test, and b represents the weight corresponding to manual evaluation, the training tasks are arranged according to scores, and the score arrangement sequence is a sequence of the recommended training tasks.

10. The user ability-based personalized cognitive training task recommendation system according to claim 6, wherein the association score wuk of the user u and the capability k is established through the following steps:
sequentially implementing n preset comprehensive evaluation subunits of the machine preliminary test by the user and respectively generating a raw score Xn;
extracting the raw score of a subject in the n comprehensive evaluation subunits, and carrying out standardized conversion on the raw scores in the comprehensive evaluation subunits according to comprehensive norm parameters of a healthy user, so as to generate the association score wuk of the user u and the capability K; the formula is as follows:
$$Wuk = wmn * \{ 100 - 10 * ( Xi - \overline{X} i ) / σ i \}$$
wherein i ranges from 1−n; Wuk represents scores of the comprehensive evaluation subunits after the standardized conversion, namely the association scores of the user u and the capability K; if the measured capability of the user is relatively poor, the more the deviation from the norm is, the larger the value of Wuk is; Xi represents the raw scores in the comprehensive evaluation subunits; X̅i represents an average value of the raw scores of the comprehensive evaluation subunits of healthy people matched with the subject by age, gender, profession and education degree; σi represents a standard deviation of the raw scores of the comprehensive evaluation subunits of the healthy people matched with the subject by age, gender, profession and education degree; X̅i and σi are the comprehensive norm parameters of the healthy user; and wmn represents the weight of the scale Im∈L to the capability kn∈K.
